(19) United States
(12) Patent Application Publication
(10) Pub. No.: US 2003/0104368 A1
(43) Pub. Date: Jun. 5, 2003

(54) Large scale protein nucleic acid interaction profiling

(76) Inventor: Kemin Zhou, Fremont, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(21) Appl. No.: 09/683,258

(22) Filed: Dec. 5, 2001

(51) Int. Cl. C12Q 1/68; G06F 19/00; G01N 33/48; G01N 33/50; C12M 1/34

(52) U.S. Cl. 435/6; 435/287.2; 702/20

(57) ABSTRACT

In one aspect of the invention, methods are provided for detecting DNA binding proteins using nucleic acid microarrays. In one embodiment, candidate fragments which are protected by DNA protein binding are detected using an oligonucleotide array.

### BACKGROUND OF INVENTION

This invention relates to genetic analysis and bioinformatics. Specifically, this invention provides methods, systems, and computer software products for large scale protein nucleic acid interaction profiling.

Regulatory elements are often only a few bases long, occupying only a negligible portion of the whole genome, but they play a critical role in defining the execution of genetic programs. Intergenic and intronic regions of the genomic sequence contain protein-binding sites that may control many essential cellular processes including transcription, replication, recombination, and DNA repair and maintenance. RNA regulatory elements regulate splicing, RNA-editing, and translation of genes. Regulatory elements in both DNA and RNA are short sequences that are extremely difficult to discover with pure computational methods. Thus, experimental methods will be necessary to discover these elements.

Recently, some attempts have been made towards obtaining information on large scale protein-nucleic acid interactions. However, published methods are limited to obtaining specific information about a particular protein or a particular gene. For example, the immunoprecipitation-based method used in two recent publications is aimed at obtaining DNA fragments that bind to particular a DNA-binding protein. See Pugh and Gilmour, Genome-wide Analysis of Protein-DNA interactions in living cells. GenomeBiology. 2 No. 4 (2001): 1013.1-.3; and Ren et al., Genome-Wide Location and Function of DNA Binding Proteins. Science. 290 (2000): 2306-9. Therefore, there is a great need in the art for large scale protein nucleic acid interaction profiling (PNIP) methods that will give a global view of the footprints of all proteins in the whole genome and are high-throughput and easy to automate.

### SUMMARY OF INVENTION

In one aspect of the invention, methods are provided for detecting the binding of a plurality of proteins with a plurality of nucleic acids. The methods include obtaining a plurality of candidate fragments from the nucleic acids; where the candidate fragments contain binding sites for the proteins and where the plurality of proteins have at least 50 proteins; and detecting the candidate fragments. The nucleic acids can be genomic DNA or RNA. The candidate fragments may be obtained by DNA foot printing technology. In one preferred embodiments, candidate fragments are determined by hybridizing them with a large number of, preferrably more than 10,000, 50,000 nucleic acid probes. The nucleic acids can be isolated or synthesized double stranded or single stranded DNA or RNA. Oligonucleotide probes are particularly preferred. The probes can be immobilized on a collection of beads or optical fibers or on a substrate.

In another aspect of the invention, methods for obtaining a profile of protein binding to the genomic DNA of a biological sample are provided. The methods include obtaining a plurality of candidate fragments from genomic DNA by eliminating unbound genomic DNA; and detecting the candidate fragments. The nucleic acids can be genomic DNA or RNA. The candidate fragments may be obtained by DNA foot printing technology. In one preferred embodiments, candidate fragments are determined by hybridizing them with a large number of, preferrably more than 10,000, 50,000, 100,000, or 1×10⁶ nucleic acid probes. The nucleic acids can be isolated or synthesized double stranded or single stranded DNA or RNA. Oligonucleotide probes are particularly preferred. The probes can be immobilized on a collection of beads or optical fibers or on a substrate.

In yet additional aspect, methods for analyzing gene expression regulation are provided. The methods include obtaining a first set of candidate fragments from the genomic DNA of a first sample, where the first sample is a control sample; obtaining a second set candidate fragments from the genomic DNA of a second sample, wherein the second sample is treated; and comparing the first and second sets of candidate fragments. The candidate fragments can be obtained using DNA foot printing technology. the second sample may be treated with a pharmaceutical agent or with an environmental change. The step of comparing candidate fragments may include hybridizing the first and second sets of candidate fragments with the same collection of nucleic acid probes. In some other embodiments, the step of comparing candidate fragments may include hybridizing the first and second sets of candidate fragments with a first and second collections of nucleic acid probes. The first and second collection of nucleic acid probes can be the same. The nucleic acid probes may be immobilized on a collection of beads or optical fibers or on a substrate. Preferably, the collection of nucleic acid probes contains at least 10,000, 50,000, 100,000, or 1,000,000 probes. The nucleic acid probes may be oligonucleotide probes, preferably between 10-50 in length. In some embodiments, the probes tile genomic sequences of interest. In preferred embodiments, at least one of the binding proteins is unknown.

### BRIEF DESCRIPTION OF DRAWINGS

he accompanying drawings, which are incorporated in and form a part of this specification, illustrate embodiments of the invention and, together with the description, serve to explain the principles of the invention:

FIG. 1 shows an exemplary process for determining protein nucleic acid interaction.

FIG. 2 shows a process for determining protein binding sites correlated with cellular states.

FIG. 3 shows a candidate fragment.

### DETAILED DESCRIPTION

Reference will now be made in detail to the preferred embodiments of the invention. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention.

All patents and publications are herein incorporated by reference in their entireties to the same extent as if each individual publication was specifically and individually indicated to be incorporated by reference.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, depending upon the context, the term “sequence” may refer to the arrangement or information content of a molecule or a molecule having the sequence.

Protein-Nucleic Acid Interaction

The interaction of proteins with DNA and RNA is central to many cellular functions. For example, protein nucleic acid interactions are involved in the packaging of chromosomes, regulation of transcription, function of ribosome and processing of RNAs.

Eukaryotic chromosomes are supra-molecular complexes of DNAs and proteins (mainly histones). They are densely packed structures depending on the stage of the cell cycle. During cell division, or mitosis, the chromosome has its highest packaging.

Transcriptional factors are another important class of proteins that bind to DNA in the regulation of gene expression. Proteins that bind DNA and are involved in replication or transcription do so in a sequence specific way. The regulation of transcription is one of the most important steps in the control of gene expression because transcription constitutes the input of the mRNA pool. One level of transcriptional control is through the binding of transcriptional factors to the cis-acting transcriptional control sequences. A human gene often employs several cis-acting sequences. Promoters are a class of cis-acting elements usually located immediately up-stream (often within 200 bp) of the transcriptional start sites. Promoters (TATA box, CCAAT Box, GC box, etc.) are often recognized by ubiquitous transcriptional factors. In addition, promoters may be involved in the control of tissue-specific expression through the binding of tissue specific transcriptional factors. Another class of cis-acting elements are the response elements (REs). Those elements are typically found in genes whose expression is responsive to the presence of signaling molecules such as growth factors, hormones, and secondary messengers. Such elements include, but not limited to, cAMP REs, retinoic acid REs, growth factor REs, glucocorticoid REs. Enhancers and repressors are yet another class of the cis-acting elements. Those elements have a positive or negative effect on transcription and their functions are generally independent of their orientation in the gene.

While there have been major advances in understanding the structures and functions of DNA binding proteins, most of the binding sites are still unknown. One difficulty in understanding protein nucleic acid interaction is the small size of the binding recognition sites. The DNA and RNA regulatory elements for regulating the transcription, splicing, and translation of genes are often very short sequences (about 20 bp) occupying less than 0.1% of the coding region and are extremely difficult to discover with computational methods.

In addition, the protein nucleic acid binding may be dynamically affected by the cellular environment, such as physiological, pharmacological and toxiological status. Therefore, experimental methods for large scale profiling of protein-nucleic acid interactions under various conditions are needed.

Overview of Large Scale Protein and Nucleic Acid Interaction Profiling

In one aspect of the invention, methods are provided for large scale protein and nucleic acid interaction profiling. The preferred methods are particularly useful for understanding the dynamic binding of regulatory proteins, such as transcriptional factors with the regulatory sequences in the genome. The methods are also useful for understanding the regulation of RNA transcriptomes.

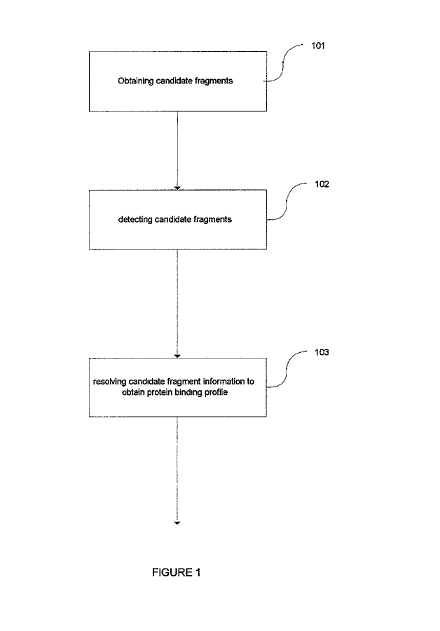

FIG. 1 shows an outline of one embodiment of the methods. The first phase is to obtain a collection of all DNA or RNA fragments (candidate fragments, CF) that contains protein binding sites (101). Nucleic acids, as used herein, may include any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

As used herein, the term “candidate fragment” refers to a nucleic acid fragment that contains information about protein nucleic acid interactions. They are sequences potentially bound by a protein or nucleic acids derived from a nucleic acid fragment that may be potentially bound by a protein. Therefore, candidate fragments may contain protein binding sequences or their complementary sequences. Candidate fragments can be any size, but are typically at least 10, 20, 30, 40 bases or base pairs and can be single or double stranded DNA or RNA. The length of a candidate fragment can vary according to the particular prtein or protein compex that binds to the site, and methods for obtaining the fragment. In preferred embodiments, the candidate fragments are obtained using footprinting technology. In such embodiments, the candidate fragments are the protected fragments.

Typically, the collection contains at least 10, 50, 100, 1000, 10,000, or 50,000 fragments. The candidate fragments are detected (102) and analyzed (103). In preferred embodiments, the candidate fragments are detected using parallel assay systems such as those using nucleic acid probe arrays, bead- or fiber-immobilized nucleic acid probes. The detection can be either qualitative or quantitative or both. For example, the sequence of the candidate fragments may be determined along with the relative level of such fragments.

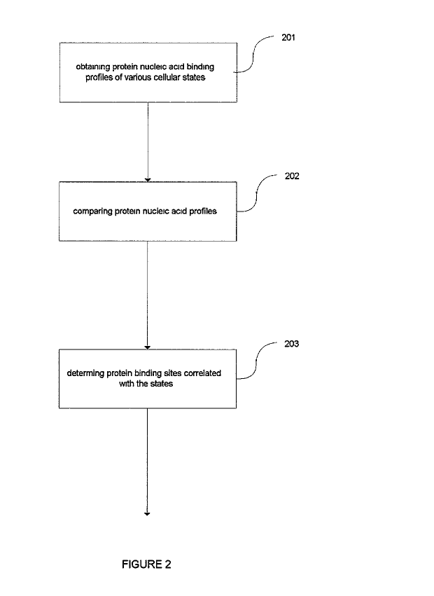

In another aspect of the invention (FIG. 2 shows one embodiment), methods, compositions and computer software are provided for analyzing protein nucleic acid binding profiles to understand the dynamic interactions. Protein-nucleic acid interaction profiles may be obtained (201) from cells of various states, such as cancer vs. normal cells, cells treated with various pharmaceutical agents, etc. The profiles may be compared (203) to discover interactions that are correlated with the states of the cells. In an exemplary embodiment, protein binding difference between diseased and normal cells may lead to potential drug targets. Various aspects of the methods and their applications will be described in the following sections in great detail using exemplary embodiments.

Biological Sample

In one aspect of the invention, biological samples reflecting different states of cells are used for protein nucleic acid interaction analysis. Such samples may be of any biological tissue or fluid or cells from any organism. Frequently the sample will be a “clinical sample” which is a sample derived from a patient. Clinical samples provide a rich source of information regarding the various states of genetic network or gene expression. Typical clinical samples include, but are not limited to, sputum, blood, blood cells (e.g., white cells), tissue or fine needle biopsy samples, urine, peritoneal fluid, and pleural fluid, or cells therefrom. Biological samples may also include sections of tissues, such as frozen sections or formalin fixed sections taken for histological purposes.

In preferred embodiments, biological samples are often obtained from cell cultures. In cell culture systems, the state of cells may be altered in a number of convenient ways to obtain samples representing a large number of independent states of gene expression. For example, cells may be treated with pharmacological or candidate pharmacological agents. In some embodiments, antisense oligonucleotides or antisense genes are used to block the expression of specific genes. In other embodiments, homozygous, knock-out techniques are used to specifically suppress the expression of genes. In other emobidments transfection of regulatory genes is used to alter the expression profile of a cell. In some additional embodiments, antisense oligonucleotides of random sequence are introduced to cells to block the expression of genes.

Obtaining Candidate Fragments

In one aspect of the invention, the method of discovering genome-wide nucleic acid-binding sites involves obtaining a set of candidate fragments of nucleic acid that a compound, such as a protein, can bind. Candidate fragments, the short nucleic acid fragments enclosed inside a region protected by a bound compound, such as a protein, may be obtained by, for example, footprinting technology. In some preferred embodiments, cells of the species of interest (the species whose genome is to be annotated, for example, E. coli., yeast, dog or human) are collected and a set of candidate fragments is prepared using methods of in vivo footprinting or, alternatively, using methods of cross-linking with UV or other chemical reagents. Additionally, protein extracts can be made in vitro from the nucleus of interested cells or tissue.

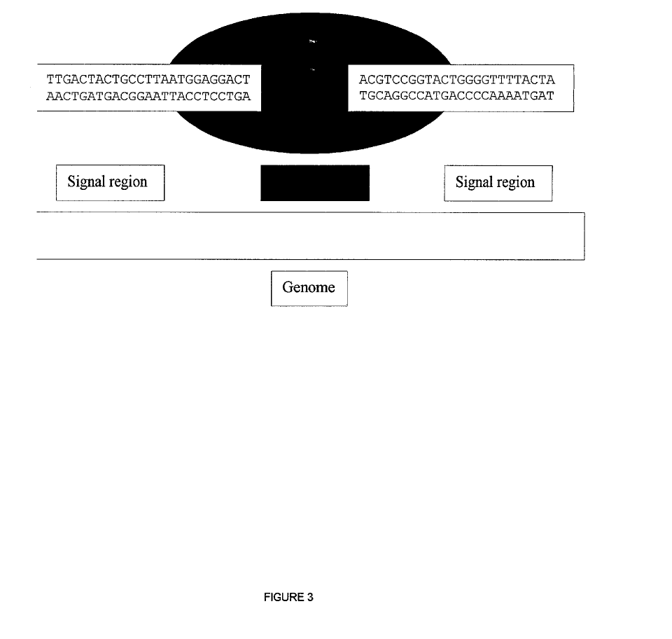

FIG. 3 is a schematic showing a candidate fragment. The fragment comes from a genome. The middle of the candidate fragment contains the binding site. In this case, the protected region (the candidate fragment) is larger than the binding site.

The candidate fragment can be defined under, for example, two different conditions: denaturing or non-denaturing relative to the proteins. Under non-denaturing conditions, the protein still binds, covalently or non-covalently, to DNA in its native form. DNase I digestion can be carried out to remove some or all unbound DNA. If the protein is crosslinked to DNA, harsher handling conditions, which usually cause protein to denature, can be used to carry out digestion of unprotected DNA. digestion can be controlled to different degrees by the average length of DNA fragments produced.

One aspect of the invention includes the use of short DNA-fragments of the size 50-500 base pair derived from a particular genome of particular cell types (such as genome-un-rearranged germline cells, or genome-rearranged cells from immune systems) by random processes such as sonication or nuclease digestion. Each method of candidate fragment preparation is discussed in detail below. Some of these method steps are well known to those of skill in the art. For example, standard methods of preparing and end-labeling DNA fragments using DNase I are described in detail in Chapter 17, Protocol 1 of Molecular Cloning: A Laboratory Manual (3^{rd} ed.), Sambrook et al., Vols. 1-3, Cold Spring Harbor Laboratory Press, New York, (2001), which is hereby incorporated herein by reference.

One of skill in the art will recognize that experimental conditions can affect collection of an appropriate set of candidate fragments. It is well known to one of ordinary skill that certain experimental conditions needs to be adjusted to account for variations of experimental conditions and objectives of the experiments. While it is not required, it is preferred to have optimal conditions to collect complex collections of candidate fragments. For some general experimental consideration, see, e.g., Rhodes and Fairall, Protein Function: A Practical Approach. IRL Press, Oxford, (1997): 215-244, which is hereby incorporated herein by reference.

Footprinting. As indicated above, one embodiment of this invention involves obtaining a set of candidate fragments through a simplified or modified version of footprinting technology. Footprinting methods have been used to obtain information about binding sequences of a particular protein to the genome or to a fragment of DNA and they are well within the skill of one of ordinary skills in the art. Footprinting can be accomplished either in vivo or in vitro. In vivo footprinting provides binding sites occupied by a protein inside a living cell, reflecting actual life conditions. Alternatively, in vitro footprinting provides binding sites under artificial conditions inside a test tube.

One version of in vivo footprint exploits the fact that most specific protein-DNA interactions have very tight binding constants. Nuclear extracts or whole cells may be obtained under certain condition or from certain tissue and lyse the cell under conditions favoring protein-DNA interaction. However, disruption of cellular organells may change the profile of protein footprints. DNA-digesting reagents, such as DNase I, can be added directly to nuclear extracts.

Another version of in vivo footprint uses cross-linking either by physical (such as UV light) or chemical (such as formaldehyde) means. Because protein and DNA or RNA is covalently linked after cross-linking, the footprint is not altered after lysing the cells. Then one skilled in the art can remove all DNA that are not protected by proteins with endo- and exon-nucleases. Next protein/DNA or protein/RNA complexes may be optionally isolated from unbound protein and DNA or RNA.

With in vitro footprinting, proteins are isolated from the nucleus or whole cells. In vitro DNA-binding reactions then bind the proteins to genomic DNA that are next processed into short DNA fragments in the range of 40-500 base pair by either enzyme digestion or sonication. The subsequent steps are similar to in vivo methods. With this method we can obtain all potential binding sites in the whole genome by all proteins in the nuclear preparation of interested cells or tissues.

Comparison of in vivo and in vitro footprint will reveal critical regulatory information. For example, in most cases, only a subset of the in vitro sites will appear in in vivo sites owing to the fact that only a small portion of the genes are expressed in a give cell type at certain time of development. Some sites that appear in in vivo footprint may not appear in vitro footprint because the binding of protein complexes to certain sites in the genome may require certain configuration of the chromatin or the native environment in the nucleus.

In vivo footprinting techniques are well-established methods and are used in numerous research papers to obtain the exact sequence of protein-binding sites without the use of any cross-linking reagents. These methods are laborious and often hazardous as large amounts of radioactive materials are required to label the DNA and sometimes introduce undesired effects.

While the traditional methods of obtaining a set of candidate fragments as protein-nucleic acid complexes are very useful for at least some embodiment of the invention, additional methods are provided for in vivo footprinting which provides a set of candidate fragments suitable for large scale protein nucleic acid interaction profiling.

In one embodiment, the methods of the present invention uses DNase I to eliminate all DNA that is not bound in the DNA-protein complex. Additionally, in one embodiment of the present invention, buffers that contain manganese (Mn+2) ions are used to prevent DNA nicking.

Additional detailed protocols for obtaining protein protected nucleic acid fragments are well known to those of skill in the art and are described in, e.g., Chapter 17 of Molecular Cloning: A Laboratory Manual (3^{rd} ed.), Sambrook et al., Vols. 1-3, Cold Spring Harbor Laboratory Press, New York, (2001); and in Unit 12.4 of Current Protocols in Molecular Biology, Fred Ausubel, Vols. 1-4, John Wiley & Sons, Inc., (1998), which are hereby incorporated herein by reference. The candidate fragments can be labeled in a number of ways. Methods of nucleic acid labeling are well known to those of ordinary skill in the art. Some labeling protocols are described in, for example, Chapters 8, 9, and 10 of Molecular Cloning: A Laboratory Manual (3^{rd} ed.), Sambrook et al., Vols. 1-3, Cold Spring Harbor Laboratory Press, New York, (2001), which is hereby incorporated herein by reference.

Some protein-nucleic acid interactions are very tight, which means that both thermodynamically and kinetically the complex is stable. Other protein-nucleic acids complexes may be kinetically less stable with faster dissociation rates and association constants. In the present invention, some protein-nucleic acid complexes will not survive the in vivo footprinting procedure without the use of a cross-linking reagent. Even in the case of a stable protein-nucleic acid complex, if the protein has a very fast degradation rate, the protein-complex will not survive very long. For example, in yeast α-cells, Mat α 2p homodimer associates with Mcm1p homodimer and binds to a 31 base pair DNA sequence to repress expression of a cell-specific gene. The protein complex has a half-life of less than five minutes. Additionally, inside a cell many protein-nucleic acid interactions are very dynamic. To capture an image of this dynamic picture, cross-linking of protein to nucleic acids is essential. Cross-linking reagents promote the formation of covalent bonds between protein and nucleic acids. Consequently, the protein-nucleic acid complex will not dissociate.

In one embodiment, a method of obtaining candidate fragments includes photo cross-linking using UV light. UV light has been successfully applied to study protein-DNA interaction. One of skill in the art will recognize that this method irradiates DNA in vivo using UV light of wavelengths in the range of 254 nm to 260 nm in order to bond compounds covalently to the DNA. However, actual protein linkage to DNA using irradiation requires several minutes and never proves to be effective. Furthermore, this extended exposure to light allows the proteins to redistribute themselves along the target DNA site eliminating accuracy of binding site locations. Standard protocols for UV crosslinking of proteins to DNA are described in Unit 12.5 of Current Protocols in Molecular Biology, Fred Ausubel, Vols. 1-4, John Wiley & Sons, Inc., (1998), which is hereby incorporated herein by reference. One advantage of this method is its scalability. Large amount of samples can be irradiated at the same time.

One of skill in the art will realize that traditional UV cross-linking has been improved with the help of lasers. Although the chemical reactions involved are not highly understood, this method is used both in vivo and in vitro to bind protein to DNA. The laser cross-linking method is very fast, inducing linkage in less than 1 μs while eliminating any redistribution of proteins. See, Angelov et al, Methods in Molecular Biology, Vol. 119: Chromatin Protocols (1999): 481-495 and Dimitrov, S. and T. Moss, UV laser-induced protein-DNA crosslinking. Methods in Molecular Biology, Vol. 30 (1994): 227-36. This method induces cross-links unable to be made under traditional methods while inducing no protein-protein complexes. More importantly, laser UV cross-linking produces a high yield 50-100 times that of the traditional method, achieving approximately 15% linkage. Additionally, cross-linking using an UV laser has the ability to capture short-lived protein-nucleic acid complexes. However, some special techniques are needed to scale up these complexes. See, Mutskov et al., A preparative method for crosslinking proteins to DNA in nuclei by single-pulse UV laser irradiation: Photochemistry Photobiology: Vol. 66 (1997):42-45.

Other crosslinking reagents such as gamma radiation and antitumor druds has also been tested. See Banjar et al., Crosslinking of chromosomal proteins to DNA in HeLa cells by UV gamma radiation and some antitumor drugs: Biochemical and Biophysical Rresearch Communications, v. 114 (1983):767-773. Formaldehyde crosslinking has also been successfully used to probe protein-DNA interaction inside living cells See See Magdinier, F. and A. P. Wolffe, Selective association of the methyl-CpG binding protein MBD2 with the silent p14/p16 locus in human neoplasia. Proceedings of the National Academy of Sciences of the United States of America, Vol. 98 (2001): 4990-4995; Schouten, J., Hybridization selection of covalent nucleic acid-protein complexes. 2. Cross-linking of proteins to specific Escherichia coli mRNAs and DNA sequences by formaldehyde treatment of intact cells. The Journal of Biological Chemistry, Vol. 260 (1985): 9929-9935; Zhang, L. and J. S. Pagano, Interferon regulatory factor 7 mediates activation of Tap-2 by Epstein-Barr virus latent membrane protein 1. Journal of Virology: Vol. 75 (2001): 341-350. A primary advantage of formaldehyde crosslinking is its reversibility; heating at 65° C. can break the covalent bond.

Another embodiment involves obtaining the candidate fragment from nucleosome bound DNA, thus relying on the DNA-binding activity of the DNA-binding proteins. This method requires making protein extracts from the nucleus of interested cells or tissues and then carrying out DNA-binding reactions of the nuclear extract with the whole genomic DNA that has been fragmented to an average of 100-500 base pairs using either enzyme digestion or sonication. Standard protocols for obtaining candidate fragments from nucleosome bound DNA are described in Brown and Fox, Methods in Molecular Biology, Vol. 90: Drug-DNA Interaction Protocols (1997): 81-95.

In one embodiment, where the compound to be bound is a protein, the labeled substrate is then mixed in vitro with the DNA-binding proteins of interest to form the desired DNA-protein complex. The protein is bound through the use of hydrogen bonds to create a sufficiently tight complex. As one of skill in the art will know, this complex will resist enzyme action, preserving the internally bound DNA fragment.

The candidate fragments as protein-nucleic acid complexes may need further purification to reveal the candidate fragment of the nucleic acid that is used to determine what portion of the genome is bound by the protein.

In one embodiment the candidate fragments as protein-nucleic acid complexes are first lysed with appropriate detergents that do not disturb the protein-nucleic acid interaction. The bound protein-nucleic acid complexes are then released as they are digested using a mild enzyme or chemical to partially digest any DNA fragments surrounding the DNA-protein complex. This purification eliminates, to some degree, uncross-linked DNA, and removes any excess cross-linked DNA that would effect the protein″s electrophorectic mobility, and prevents the coupling of multiple sub-protein units. See, e.g., Kaplan and Sorger, Protein Function: A Practical Approach, IRL Press, Oxford, (1997): 245-278. The most common enzymes and chemicals used in digestion are DNase I, Dimethylsulfate (DMS), and the hydroxyl radical. See, e.g., Rhodes and Fairall, Protein Function: A Practical Approach, IRL Press, Oxford, (1997): 215-244.

DNase I is the most frequently applied endonuclease in footprinting technology. DNase I is initially purified from beef pancreas. It cleaves to both double-stranded and single-stranded DNA. Cleavage preferentially occurs adjacent to pyrimidine residues. DNase I is an endonuclease, meaning cleavage can occur anywhere in the DNA molecule. Major products are 5″-phosphorylated di-, tri- and tetranucleotides. In the presence of magnesium ions (Mg2+), DNase I hydrolyzes each strand of duplex DNA independently, generating random cleavages. In the presence of manganese ions (Mn2+), the enzyme cleaves both strands of DNA at approximately the same site, producing blunt ends or fragments with 1 -2 base overhangs. DNase I does not cleave RNA. Genomic DNA that is protected by sequence-specific DNA-binding proteins is not accessible to DNase I and thus left undigested.

DNase I can diffuse into the nucleus when added to preparations of isolated nuclei. Another way of delivery is through endogenous expression of DNase I inside the cell. This has been successfully applied to Sacchromyces cerevisiae. See Wang, X. and R. T. Simpson, Chromatin structure mapping in Saccharomyces cerevisiae in vivo with DNase I. Nucleic Acids Research, Vol. 29 (2001):1943-1950. The advantage of the in vivo expression of DNase I is that it eliminates the perturbation of the protein-DNA binding state induced during the process of nuclei isolation.

Unlike DNase I, the hydroxyl radical is very small. The hydroxyl radical″s smaller size allows it to cut DNA anywhere on the sequence. However, because the hydroxyl radical can cleave the DNA strand anywhere, the rates of cleavage of the DNA-protein complex and the naked DNA are very similar making it harder to obtain a candidate fragment. Additionally, the small size of the radical prohibits an efficient cleavage reaction. Thus, to improve results when using this nuclease, one of skill in the art must use unnicked DNA fragments.

DMS is another small molecule that sharply cleaves DNA. DMS methylates guanine bases and cleaves the DNA by eliminating the mehtylated base and heating in the solution piperidine. However, DNS is only effective where guanine lies in the sequence to be cut. Additionally, the concentration of DMS required to efficiently react depends on the amount of protein and DNA. Thus, where there are large amounts of DNA, competitor DNA, and protein, a larger amount of DMS is required to efficiently cleave unwanted DNA fragments.

Similarly micrococcal nuclease that cuts linker DNA almost exclusively can be used to probe the nucleosomal structure of nuclear genomes. The principle of the methods depends on the steric hindrance or accessibility of their substrates.

Non-enzymatic organic molecules have also been developed for probing protein-DNA interactions, such as [Fe(II)(EDTA)]²⁻ and (1,4,7-trimethyl-1,4,7-triazacyclononane)iron(III) (short name L″FeCl₃). See Ehmann et al., (1,4,7-trimethyl-1,4,7-triazacyclononane)iron(III)-mediated cleavage of DNA: detection of selected protein-DNA interactions. Nucleic Acids Research, Vol. 26 (1998): 2086-2091; and Wang, X. and R. T. Simpson, Chromatin structure mapping in Saccharomyces cerevisiae in vivo with DNase I. Nucleic Acids Research, Vol. 29 (2001): 1943-1950. Cleavage of DNA by L″FeCl3 is protected by sequence-specific DNA-binding proteins, whereas cleavage of DNA by [Fe(II)(EDTA)]²⁻ is nucleosomal specific. The later acts are accomplished through the use of hydroxyl radicals. See Zaychikov, et al., Hydroxyl radical footprinting: Methods in Molecular Biology, Vol. 148 (2001):49-61. Other nucleic acid cleaving reagents include 1,10-phenanthroline-copper (Papavassiliou, A. G., Footprinting DNA-protein interactions in native polyacrylamide gels by chemical nucleolytic activity of 1,10-phenanthroline-copper. Methods in Molecular Biology, Vol. 148 (2001): 77-110), uranyl ion (UO22+) (Nielsen, P. E., Uranyl photofootprinting. Methods in Molecular Biology, Vol. 148 (2001):111-9), and osmium tetraoxide (McClellan, J. A., Osmium tetroxide modification and the study of DNA-protein interactions. Methods in Molecular Biology, Vol. 148 (2001): 121-34).

In another embodiment, the bound DNA-protein complexes are treated with sonication. Sonication uses high-frequency sound waves to break the non-bound portions of the DNA strands. L. Stryer, Biochemistry, 4^{th} Ed., W. H. Freeman and Co., New York, (March 1995): 271. Standard protocols for sonication are described in Chapter 12, Protocol 1 of Molecular Cloning: A Laboratory Manual (3^{rd} ed.), Sambrook et al., Vols. 1-3, Cold Spring Harbor Laboratory Press, NY, (2001). Additional protocols for the sonication of DNA specifically are described by Richard Young at Genome-wide Location and Function of DNA Binding Proteins. Richard Young. (2000). Massachusettes Institute of Technology. Jun. 25, 2001 <http://web.wi.mit.edu/young/location>.

In some embodiments, the protein-nucleic acid complexes need to be isolated from unbound DNA in the sample. If the DNA is not crosslinked to protein, the isolation condition has to favor the protein-nucleic acid interaction. If the protein is crosslinked to nucleic acids, very harsh conditions can be used for the isolation. There are many ways to isolate protein-DNA complex from the mixture of protein and DNA.

In one embodiment, DNA fragments that are cross-linked to protein can be easily isolated from free DNA fragments using phenol extractions. Free DNA will be partitioned into the aqueous phase, whereas the protein-DNA complex will be in the phenol and interface. See Belikov, et al., Two non-histone proteins are associated with the promoter region and histone HI with the transcribed region of active hsp-70 genes as revealed by UV-induced DNA-protein crosslinking in vivo. Nucleic Acids Research, Vol. 21 (1993): 1031-1034. Under DNA-denaturing conditions, the strand to which protein attach to can be resolved. Related protocols for phenol extraction are rd described in Chapter 7, Protocol 1 of Molecular Cloning: A Laboratory Manual (3^{rd} ed.), Sambrook et al., Vols. 1-3, Cold Spring Harbor Laboratory Press, New York, (2001). Related protocols for membrane filtration are described in Chapter 7, Protocol rd 7 of Molecular Cloning: A Laboratory Manual (3^{rd} ed.), Sambrook et al., Vols. 1-3, Cold Spring Harbor Laboratory Press, New York, (2001).

In another embodiment, nitrocellulose filters have the property of binding protein-nucleic acids complexes but letting free nucleic acids pass through. See Stockley, P. G., Filter-binding assays. Methods in Molecular Biology. Vol. 148 (2001): 1-11. The filter binding method is rapid and reproducible. In the present embodiment it is not necessary to label the ends of the DNA before filtering.

In another embodiment in which an interest is focused on particular proteins, the protein-nucleic acid complexes are collected through immunoprecipitation. Immunoprecipitation is particularly useful when partitioning the protein-nucleic acid complex or eliminating unwanted protein-nucleic complexes. Immunoprecipitation is a method in which an antigen, such as a protein or a DNA/protein complex, is isolated by binding to a specific antibody. As one skilled in the art will know, immunoprecipitation can be used positively to obtain the proteins of interest, or it can be used negatively to eliminate unwanted histomes and other undesired compounds.

As one of skill in the art will know, the process of immunoprecipitation involves three major steps. First, the antigen, or protein, is solubilized by lysing the cell using either non-denaturing detergents or under denaturing conditions. One skilled on the art will know that this simple cell lysing is suitable for animal cells. Yeast cells, however, require their cell wall to be physically damaged before extraction of the antigens. Second, the antibody binds either noncovalently to protein A- or protein G-agarose beads or covalently to Sepharose to immobilize the antibody. Finally, the antigen is captured as it is isolated on the antibody-conjugated beads. Standard protocols for immunoprecipitation are described in Unit 10.16 of Current Protocols in Molecular Biology, Fred Ausubel, Vols. 1-4, John Wiley & Sons, Inc., (1998), which is hereby incorporated herein by reference.

Before the candidate fragments are analyzed, it is often desirable to separate them from the proteins. In some instances, the candidate fragments may be separated from their binding proteins and the proteins are then removed. In some other instances, proteins in the protein-nucleic acid complexes may be removed by digestion with proteases. One of skill in the art would appreciate that the methods of the invention are not limited to any particular protease or combination of protease. Rather, any suitable protease may be used. In one preferred embodiment, proteinase K is used for the digestion. In some cases, chemical digestion may also be used to remove the proteins. It is worth noting that if the proteins are cross-linked, some residual amino acids left on the nucleic acids would not interfere with further analysis and therefore, it is not required to remove all amino acids before further analysis can be pursued.

Candidate Fragment Detection

The collection of candidate fragments contains information of not only what regions of the genome or transcriptome are occupied, but also the frequency of occupancy. Because the set of candidate fragments is very large, especially with eukaryotic genomes, it is preferred to use very high capacity methods such as nucleic acid microarray, nucleic acid immobilized beads or optical fibers.

High density nucleic acid probe arrays, also referred to as DNA Microarrays, have become a method of choice for monitoring the expression of a large number of genes and for detecting sequence variations, mutations and polymorphism.

In preferred embodiments, probes may be immobilized on substrates to create an array. An array may comprise a solid support with peptide or nucleic acid or other molecular probes attached to the support. Arrays typically comprise a plurality of different nucleic acids or peptide probes that are coupled to a surface of a substrate different, known locations. These arrays, also described as microarrays or colloquially chips have been generally described in the art, for example, in Fodor et al., Science, 251:767-777 (1991), which is incorporated by reference for all purposes. Methods of forming high density arrays of oligonucleotides, peptides and other polymer sequences with a minimal number of synthetic steps are disclosed in, for example, U.S. Pat. Nos. 5,143,854, 5,252,743, 5,384,261, 5,405,783, 5,424,186, 5,429,807, 5,445,943, 5,510,270, 5,677,195, 5,571,639, 6,040,138, all incorporated herein by reference for all purposes. The oligonucleotide analogue array can be synthesized on a solid substrate by a variety of methods, including, but not limited to, light-directed chemical coupling, and mechanically directed coupling. (See Pirrung et al., U.S. Pat. No. 5,143,854, PCT Application No. WO 90/15070) and Fodor et al., PCT Publication Nos. WO 92/10092 and WO 93/09668, U.S. Pat. Nos. 5,677,195, 5,800,992 and 6,156,501, which disclose methods of forming vast arrays of peptides, oligonucleotides and other molecules using, for example, light-directed synthesis techniques.) (See also Fodor, et al., Science, 251, 767-77 (1991)). These procedures for synthesis of polymer arrays are now referred to as VLSIPS™ procedures.

Methods for making and using molecular probe arrays, particularly nucleic acid probe arrays are also disclosed in, for example, U.S. Pat. Nos. 5,143,854, 5,242,974, 5,252,743, 5,324,633, 5,384,261, 5,405,783, 5,409,810, 5,412,087, 5,424,186, 5,429,807, 5,445,934, 5,451,683, 5,482,867, 5,489,678, 5,491,074, 5,510,270, 5,527,681, 5,527,681, 5,541,061, 5,550,215, 5,554,501, 5,556,752, 5,556,961, 5,571,639, 5,583,211, 5,593,839, 5,599,695, 5,607,832, 5,624,711, 5,677,195, 5,744,101, 5,744,305, 5,753,788, 5,770,456, 5,770,722, 5,831,070, 5,856,101, 5,885,837, 5,889,165, 5,919,523, 5,922,591, 5,925,517, 5,658,734, 6,022,963, 6,150,147, 6,147,205, 6,153,743 and 6,140,044, all of which are incorporated by reference in their entireties for all purposes.

Microarray can be used in a variety of ways. A preferred microarray contains nucleic acids and is used to analyze nucleic acid samples. Typically, a nucleic acid sample is prepared from appropriate source and labeled with a signal moiety, such as a fluorescent label. The sample is hybridized with the array under appropriate conditions. The arrays are washed or otherwise processed to remove non-hybridized sample nucleic acids. The hybridization is then evaluated by detecting the distribution of the label on the chip. The distribution of label may be detected by scanning the arrays to determine fluorescence intensity distribution. Typically, the hybridization of each probe is reflected by several pixel intensities. The raw intensity data may be stored in a gray scale pixel intensity file. The GATC™ Consortium has specified several file formats for storing array intensity data. The final software specification is available at www.gatcconsortium.org and is incorporated herein by reference in its entirety. The pixel intensity files are usually large. For example, a GATC™ compatible image file may be approximately 50 Mb if there are about 5000 pixels on each of the horizontal and vertical axes and if a two byte integer is used for every pixel intensity. The pixels may be grouped into cells. (See GATC™ software specification). The probes in a cell are designed to have the same sequence; i.e., each cell is a probe area. A CEL file contains the statistics of a cell, e.g., the 75th percentile and standard deviation of intensities of pixels in a cell. The 50, 60, 70, 75 or 80th percentile of pixel intensity of a cell is often used as the intensity of the cell.

The Affymetrix® Analysis Data Model (AADM) is the relational database schema Affymetrix uses to store experiment results. It includes tables to support mapping, spotted arrays and expression results. Affymetrix publishes AADM to support open access to experiment information generated and managed by Affymetrix® software that results may be filtered and mined with any compatible analysis tools. The AADM specification (Affymetrix, Santa Clara, Calif., 2001) is incorporated herein by reference for all purposes. The specification is available at http://www.affymetrix.com/support/aadm/aadm.html, last visited on Sep. 4, 2001.

Methods for signal detection and processing of intensity data are additionally disclosed in, for example, U.S. Pat. Nos. 5,445,934, 547,839, 5,578,832, 5,631,734, 5,800,992, 5,856,092, 5,936,324, 5,981,956, 6,025,601, 6,090,555, 6,141,096, 6,141,096, and 5,902,723. Methods for array based assays, computer software for data analysis and applications are additionally disclosed in, e.g., U.S. Pat. Nos. 5,527,670, 5,527,676, 5,545,531, 5,622,829, 5,631,128, 5,639,423, 5,646,039, 5,650,268, 5,654,155, 5,674,742, 5,710,000, 5,733,729, 5,795,716, 5,814,450, 5,821,328, 5,824,477, 5,834,252, 5,834,758, 5,837,832, 5,843,655, 5,856,086, 5,856,104, 5,856,174, 5,858,659, 5,861,242, 5,869,244, 5,871,928, 5,874,219, 5,902,723, 5,925,525, 5,928,905, 5,935,793, 5,945,334, 5,959,098, 5,968,730, 5,968,740, 5,974,164, 5,981,174, 5,981,185, 5,985,651, 6,013,440, 6,013,449, 6,020,135, 6,027,880, 6,027,894, 6,033,850, 6,033,860, 6,037,124, 6,040,138, 6,040,193, 6,043,080, 6,045,996, 6,050,719, 6,066,454, 6,083,697, 6,114,116, 6,114,122, 6,121,048, 6,124,102, 6,130,046, 6,132,580, 6,132,996 and 6,136,269, all of which are incorporated by reference in their entireties for all purposes.

Nucleic acid probe array technology, use of such arrays, analysis array based experiments, associated computer software, composition for making the array and practical applications of the nucleic acid arrays are also disclosed, for example, in the following U.S. patent application Ser. Nos. 07/838,607, 07/883,327, 07/978,940, 08/030,138, 08/082,937, 08/143,312, 08/327,522, 08/376,963, 08/440,742, 08/533,582, 08/643,822, 08/772,376, 09/013,596, 09/016,564, 09/019,882, 09/020,743, 09/030,028, 09/045,547, 09/060,922, 09/063,311, 09/076,575, 09/079,324, 09/086,285, 09/093,947, 09/097,675, 09/102,167, 09/102,986, 09/122,167, 09/122,169, 09/122,216, 09/122,304, 09/122,434, 09/126,645, 09/127,115, 09/132,368, 09/134,758, 09/138,958, 09/146,969, 09/148,210, 09/148,813, 09/170,847, 09/172,190, 09/174,364, 09/199,655, 09/203,677, 09/256,301, 09/285,658, 09/294,293, 09/318,775, 09/326,137, 09/326,374, 09/341,302, 09/354,935, 09/358,664, 09/373,984, 09/377,907, 09/383,986, 09/394,230, 09/396,196, 09/418,044, 09/418,946, 09/420,805, 09/428,350, 09/431,964, 09/445,734, 09/464,350, 09/475,209, 09/502,048, 09/510,643, 09/513,300, 09/516,388, 09/528,414, 09/535,142, 09/544,627, 09/620,780, 09/640,962, 09/641,081, 09/670,510, 09/685,011, and 09/693,204 and in the following Patent Cooperative Treaty (PCT) applications/publications: PCT/NL90/00081, PCT/GB91/00066, PCT/US91/08693, PCT/US91/09226, PCT/US91/09217, WO/93/10161, PCT/US92/10183, PCT/GB93/00147, PCT/US93/01152, WO/93/22680, PCT/US93/04145, PCT/US93/08015, PCT/US94/07106, PCT/US94/12305, PCT/GB95/00542, PCT/US95/07377, PCT/US95/02024, PCT/US96/05480, PCT/US96/11147, PCT/US96/14839, PCT/US96/15606, PCT/US97/01603, PCT/US97/02102, PCT/GB97/005566, PCT/US97/06535, PCT/GB97/01148, PCT/GB97/01258, PCT/US97/08319, PCT/US97/08446, PCT/US97/10365, PCT/US97/17002, PCT/US97/16738, PCT/US97/19665, PCT/US97/20313, PCT/US97/21209, PCT/US97/21782, PCT/US97/23360, PCT/US98/06414, PCT/US98/01206, PCT/GB98/00975, PCT/US98/04280, PCT/US98/04571, PCT/US98/05438, PCT/US98/05451, PCT/US98/12442, PCT/US98/12779, PCT/US98/12930, PCT/US98/13949, PCT/US98/15151, PCT/US98/15469, PCT/US98/15458, PCT/US98/15456, PCT/US98/16971, PCT/US98/16686, PCT/US99/19069, PCT/US98/18873, PCT/US98/18541, PCT/US98/19325, PCT/US98/22966, PCT/US98/26925, PCT/US98/27405 and PCT/IB99/00048, all the above cited patent applications and other references cited throughout this specification are incorporated herein by reference in their entireties for all purposes.

One of skill in the art will appreciate that an enormous number of array designs are suitable for the practice of this invention. The high density array will typically include a number of probes that specifically hybridize to the candidate fragments of interest. In addition, in a preferred embodiment, the array will include one or more control probes.

The high density array chip includes test probes. Test probes could be oligonucleotides that range from about 5 to about 45 or 5 to about 500 nucleotides, more preferably from about 10 to about 40 nucleotides and most preferably from about 15 to about 40 nucleotides in length. In other particularly preferred embodiments the probes are 20 or 25 nucleotides in length. In another preferred embodiment, test probes are double or single strand DNA sequences. DNA sequences are isolated or cloned from nature sources or amplified from nature sources using nature nucleic acid as templates. These probes have sequences complementary to particular subsequences of the genes whose expression they are designed to detect. Thus, the test probes are capable of specifically hybridizing to the target nucleic acid they are to detect.

The probes for detecting candidate fragments may be selected in a number of ways. In one particular implementation, probes are selected to tile a large subsection or the entire genome. In other implementations, probes may be selected to detect candidate fragments from interested regions. For example, if one is interested in understanding the regulation of the expression of a group genes, probes may be selected to detect (or to be complementary with) sub-regions of the genes.

As genome annotation progresses, a portions of the genome where binding to regulatory elements is more likely can be put on the probe arrays. For example, sequences in inter-genenic regions and large introns (longer than 1 kb in human, not so many introns are longer than 1 kb) with repeats masked. Both the forward and the lower strand sequence may be represented on the chip; this is essential for eliminated false positives.

In addition to the nucleic acid array based methods, other methods may also be used to determine the candidate fragments. For example, massive parallel sequence approach (MPS)(Lynx) may also be used.

The labeled candidate fragments are then hybridized with appropriate probes on a microarray to yield the sequences and binding sites of the compound on the nucleic acid fragment. Nucleic acid hybridization simply involves contacting a probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches. One of skill in the art will appreciate that hybridization conditions may be selected to provide any degree of stringency.

Data Analysis In one embodiment, once the sets of candidate fragments are collected, the profile can be analyzed. The set of all candidate fragments represents a snap shot of the genome that is occupied by proteins. The intensity of candidate fragment sequences represents the relative frequency that each site is occupied. How the footprint and profile changes under different conditions or during development will reveal critical regulatory information on a whole system level. This invention thus provides a whole new field of information collection and analysis regarding cellular regulation.

The methods of the invention are powerful tool with extensive applications in areas such as drug discovery. For example, protein nucleic acid interaction profiles may be obtained from normal and cancer cells. The profiles can be compared to discover differences of the cells in terms of chromatin structure and the sequence-specific protein-binding sites on the genome. Similarly, biologist can compare and detect all the protein-binding sites at a particular developmental stage, disease condition, drug treatment, etc.

As will be appreciated by one of skill in the art, the present invention may be embodied as a method, data processing system or program products. Accordingly, the present invention may take the form of data analysis systems, methods, analysis software and etc. Software written according to the present invention is to be stored in some form of computer readable medium, such as memory, hard-drive, DVD ROM or CD ROM, or transmitted over a network, and executed by a processor.

All publications and patent applications cited above are incorporated by reference in their entirety for all purposes to the same extent as if each individual publication or patent application were specifically and individually indicated to be so incorporated by reference. Although the present invention has been described in some detail by way of illustration and example for purposes of clarity and understanding, it will be apparent that certain changes and modifications may be practiced within the scope of the appended claims.

1. A method for detecting the binding of a plurality of proteins with a plurality of nucleic acids comprising:
a. obtaining a plurality of candidate fragments from the nucleic acids; wherein the candidate fragments contain binding sites for the proteins and wherein the plurality of proteins have at least 50 proteins; and
b. detecting the candidate fragments.

2. The method of claim 1, wherein the nucleic acid is DNA.

3. The method of claim 2 wherein the nucleic acid is genomic DNA.

4. The method of claim 3 wherein the candidate fragments are obtained by DNA foot printing.

5. The method of claim 4 wherein the step of determining candidate fragments comprises hybridizing the candidate fragments with a collection of nucleic acid probes.

6. The method of claim 5 wherein the nucleic acid probes are immobilized on a collection of beads or optical fibers.

7. The method of claim 5 wherein the nucleic acid probes are immobilized on a substrate.

8. The method of claim 7 wherein the collection of nucleic acid probes contain at least 10,000 probes.

9. The method of claim 8 wherein the collection of nucleic acid probes contain at least 50,000 probes.

10. The method of claim 9 wherein the collection of nucleic acid probes contain at least 100,000 probes.

11. The method of claim 10 wherein the collection of nucleic acid probes contain at least 1,000,000 probes.

12. The method of claim 10 wherein the nucleic acid probes are oligonucleotide probes.

13. The method of claim 12 wherein the oligonucleotide probes are between 10-50 in length.

14. The method of claim 13 wherein the oligonucleotide probes tile genomic sequences of interest.

15. The method of claim 14 wherein the genomic sequences of interest contain genic regions.

16. The method of claim 14, where the forward and lower strand sequences are tiled.

17. The method of claim 15 wherein at least one of the binding proteins is unknown.

18. A method for obtaining a profile of protein binding to the genomic DNA of a biological sample comprising:
a. obtaining a plurality of candidate fragments from genomic DNA by eliminating unbound genomic DNA; and
b. detecting the candidate fragments.

19. The method of claim 18, wherein the candidate fragments are obtained by DNA foot printing.

20. The method of claim 19 wherein the step of determining candidate fragments comprises hybridizing the candidate fragments with a collection of nucleic acid probes.

21. The method of claim 20 wherein the nucleic acid probes are immobilized on a collection of beads or optical fibers.

22. The method of claim 20 wherein the nucleic acid probes are immobilized on a substrate.

23. The method of claim 22 wherein the collection of nucleic acid probes contains at least 10,000 probes.

24. The method of claim 23 wherein the collection of nucleic acid probes contains at least 50,000 probes.

25. The method of claim 24 wherein the collection of nucleic acid probes contains at least 100,000 probes.

26. The method of claim 25 wherein the collection of nucleic acid probes contains at least 1,000,000 probes.

27. The method of claim 26 wherein the nucleic acid probes are oligonucleotide probes.

28. The method of claim 27 wherein the oligonucleotide probes are between 10-50 in length.

29. The method of claim 28 wherein the oligonucleotide probes tile genomic sequences of interest.

30. The method of claim 29 wherein the genomic sequences of interest contain genic regions.

31. The method of claim 29, where the forward and lower strand sequences are tiled.

32. The method of claim 31 wherein at least one of the binding proteins is unknown.

33. A method for analyzing gene expression regulation comprising:
a) obtaining a first set of candidate fragments from the genomic DNA of a first sample, wherein the first sample is a control sample;
b) obtaining a second set candidate fragments from the genomic DNA of a second sample, wherein the second sample is treated; and
c) comparing the first and second sets of candidate fragments.

34. The method of claim 33 wherein the candidate fragments are obtained by DNA foot printing.

35. The method of claim 34 wherein the second sample is treated with a pharmaceutical agent.

36. The method of claim 34 wherein the second sample is treated with environmental change.

37. The method of claim 36 wherein the step of comparing candidate fragments comprises hybridizing the first and second sets of candidate fragments with the same collection of nucleic acid probes.

38. The method of claim 37 wherein the step of comparing candidate fragments comprises hybridizing the first and second sets of candidate fragments with a first and second collections of nucleic acid probes.

39. The method of claim 38 wherein the first and second collection of nucleic acid probes are the same.

40. The method of claim 37, 38 or 39 wherein the nucleic acid probes are immobilized on a collection of beads or optical fibers.

41. The method of claim 37, 38 or 39 wherein the nucleic acid probes are immobilized on a substrate.

42. The method of claim 41 wherein the collection of nucleic acid probes contains at least 10,000 probes.

43. The method of claim 42 wherein the collection of nucleic acid probes contains at least 50,000 probes.

44. The method of claim 43 wherein the collection of nucleic acid probes contains at least 100,000 probes.

45. The method of claim 44 wherein the collection of nucleic acid probes contains at least 1,000,000 probes.

46. The method of claim 42 wherein the nucleic acid probes are oligonucleotide probes.

47. The method of claim 46 wherein the oligonucleotide probes are between 10-50 in length.

48. The method of claim 47 wherein the oligonucleotide probes tile genomic sequences of interest.

49. The method of claim 48 wherein the genomic sequences of interest contain genic regions.

50. The method of claim 49 where the forward and lower strand sequences are tiled.

51. The method of claim 50 wherein at least one of the binding proteins is unknown.
